(19) United States
(12) Patent Application Publication
(10) Pub. No.: US 2008/0171400 A1
(43) Pub. Date: Jul. 17, 2008

(54) MAGNETIC MICROPARTICLE-PACKING UNIT, MICROFLUIDIC DEVICE INCLUDING THE SAME, AND IMMUNOASSAY METHOD USING THE MICROFLUIDIC DEVICE

(75) Inventors: Yoon-kyoung CHO, Yongin-si (KR); Beom-seok LEE, Yongin-si (KR); Jeong-gun LEE, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD, Suwon-si (KR)

(21) Appl. No.: 11/839,023

(22) Filed: Aug. 15, 2007

(30) Foreign Application Priority Data
Aug. 30, 2006 (KR) 2006-0082941

(51) Int. Cl. G01N 33/553; B01J 19/00; G01N 33/552

(52) U.S. Cl. 436/526; 422/243; 436/527

(57) ABSTRACT

A magnetic microparticle-packing unit using centrifugal force, a microfluidic device including the same, and an immunoassay method using the microfluidic device are provided. The magnetic microparticle-packing unit includes a rotary body controllably rotating; a microfluidic channel which includes a curved portion in which the microfluidic channel first extends away from the rotation center of the rotary body and then turns toward the rotation center of the rotary body.

### CROSS-REFERENCE TO RELATED PATENT APPLICATION

This application claims the benefit of Korean Patent Application No. 10-2006-0082941, filed on Aug. 30, 2006, in the Korean Intellectual Property Office, the disclosure of which is incorporated herein in its entirety by reference.

### BACKGROUND OF THE INVENTION

1. Field of the Invention

The present invention relates to a magnetic microparticle-packing unit using centrifugal force, a microfluidic device including the same, and an immunoassay method using the microfluidic device, and more particularly, to a microfluidic device which can effectively pack magnetic microparticles and an immunoassay method using the microfluidic device.

2. Description of the Related Art

An immunoassay is a test including marking any one or both of an antigen and an antibody with a radioisotope, a chemiluminescent material, or the like and measuring the existence of the antigen or the antibody through an antigen-antibody reaction. A method using the radioisotope is known as a radioimmunoassay, and a method using the chemiluminescent material is known as an immunofluorescence.

Recently, research has been conducted to perform such an immunoassay using a biochip, a biosensor, a microfluidic device, or the like, which are fabricated using a minute fabrication technology. In order to perform an immunoassay based on a solid surface using microparticles in a microfluidic device, it is necessary to pack the microparticles into a predetermined space such as a channel or a chamber, through which a sample flows. Therefore, a structure is needed to contain a number of microparticles in the microfluidic device. Recently, research has been conducted into ways of performing various operations while moving fluid in a compact disc (CD)-shaped microfluidic device using centrifugal force. A device capable of effectively packing microparticles using centrifugal force is needed in order to perform an immunoassay based on a solid surface using microparticles in such a CD-shaped microfluidic device.

When a microfluidic device is packed with microparticles of which a diameter is as small as those commercially available, it is difficult to fabricate a structure to contain the microparticles. In addition, fluid pressure in the microfluidic device is greatly reduced as the fluid passes through the packed microparticles. In particular, in a CD-shaped microfluidic device, the fluid pressure loss causes a necessity of increasing the rotation speed of the device in order to transfer the fluid. On the other hand, the use of larger microparticles can reduce the pressure loss, but the gap between the packed microparticles is increased, lowering the efficiency of an immune reaction.

### SUMMARY OF THE INVENTION

The present invention provides a magnetic microparticle-packing unit and a microfluidic device having the same capable of packing magnetic microparticles using centrifugal force and magnetic force in a microfluidic structure disposed on a rotary body.

The present invention also provides a method of performing an immunoassay using the microfluidic device and a magnetic microparticle having a size adequate for packing.

According to an aspect of the present invention, there is provided a magnetic microparticle-packing unit including: a rotary body controllably rotating; a microfluidic channel disposed on the rotary body, and a magnet, wherein the microfluidic channel includes an inlet, an outlet, a curved portion, a first flow passage formed between the inlet and the curved portion, and a second flow passage formed between the curved portion and the outlet, wherein the inlet, the outlet, the curved portion, the first flow passage and the second flow passage each are in fluid communication with the other; wherein a distance from the rotation center of the rotary body to the inlet is smaller than that from the rotation center of the rotary body to the outlet, and a flow direction in the flow passage is from the inlet to the outlet; wherein the curved portion is formed in such a way that the flow passage first extends away from the rotation center of the rotary body and then turns toward the rotation center of the rotary body; and wherein the magnet is disposed so as to apply a magnetic force to the curved portion.

The microfluidic channel may further include a bottleneck portion in which the internal dimension of the flow passage is reduced. The bottleneck portion may be disposed at a rear end portion of the curved portion along the flow passage, i.e., between the curved portion and the second flow passage. The magnet may be affixed to the rotary body at a position adjacent to the curved portion or be formed in the shape of a ring having a radius corresponding to the distance from the rotation center of the rotary body to the curved portion, and is disposed outside the rotary body along the rotation trace of the curved portion. The curved portion may be U-shaped or V-shaped such that an outer boundary of the curved portion faces a rim of the rotating body.

The rotary body may have various shapes adequate for rotation, for example, be a compact disc-shaped rotating plate. The rotary body may be comprised of a upper layer and a lower layer, which are joined together by way of an adhesive, an adhesive layer, or heat sealing, in such a way to define the microfluidic channel. According to another aspect of the present invention, there is provided a microfluidic device including: a rotary body which controllably rotates and comprises a rotation center and a periphery; a microfluidic channel disposed on the rotary body, said microfluidic channel comprising an inlet, an outlet, a curved portion, a first flow passage formed between the inlet and the curved portion, and a second flow passage formed between the curved portion and the outlet, wherein the inlet, the outlet, the curved portion, the first flow passage and the second flow passage each are in fluid communication with the other; wherein a distance from the rotation center of the rotary body to the inlet is smaller than that from the rotation center of the rotary body to the outlet, and a flow direction in the flow passage is from the inlet to the outlet; and wherein the curved portion is formed in such a way that the flow passage first extends away from the rotation center of the rotary body and then turns toward the rotation center of the rotary body; and a magnet which is disposed so as to apply a magnetic force to the curved portion; a first chamber which is in fluid communication with the microfluidic channel through the inlet of the microfluidic channel; and a second chamber which is in fluid communication with the microfluidic channel through the outlet of the microfluidic channel. The microfluidic channel may further comprise a bottleneck portion in which the internal dimension of the flow passage is reduced at a position close to the curved portion and the bottleneck portion may be disposed at a rear end portion of the curved portion along the flow passage. That is, the bottleneck portion may be situated between the curved portion and the second flow passage. The magnet may be affixed to the rotary body at a position adjacent to the curved portion or be formed in the shape of a ring having a radius corresponding to the distance from the rotation center of the rotary body to the curved portion outside the rotary body along the rotation trace of the curved portion. The curved portion may be U-shaped or V-shaped such that an outer portion of the curved portion faces the periphery of the rotating body. The rotary body may have various shapes adequate for rotation, for example, be a compact disc-shaped rotating plate.

According to another aspect of the present invention, there is provided an immunoassay method using the microfluidic device. The immunoassay method according to present invention includes: introducing a fluid sample to be assayed for a target substance and magnetic microparticles into the microfluidic device, said magnetic microparticles being capable of capturing the target substance; and allowing the magnetic microparticles be contact with the fluid sample in the microfluidic device, wherein the fluid sample and the magnetic microparticles are contacted to each other in the first chamber, the magnetic microparticles are packed in the curved portion; and the fluid sample is discharged through the outlet.

The diameter of the magnetic microparticle may range from 10 to 50 μm. The magnetic microparticles may include a core formed of a magnetic material and a shell or a coating formed on the surface of the core. The shell may be formed of a nonmagnetic material. The shell may be formed of a biologically inert polymer material and a surface of the shell may be biologically activated.

In more details the shell may be formed of any one material selected from the group consisting of styrenes agarose, dextran, and polyethylene glycol (PEG). The surface of the shell may be modified such that predetermined biomolecules are specifically bound thereto. The surface of the shell may have at least one probe selected from the group consisting of an antibody, antigen, nucleic acid, biotin, protein, amino group (NH₂—), and carboxyl group (COOH—). The surface of the shell may be modified by treating it with silica.

### BRIEF DESCRIPTION OF THE DRAWINGS

The above and other features and advantages of the present invention will become more apparent by describing in detail exemplary embodiments thereof with reference to the attached drawings in which:

FIGS. 1A and 1B are plan views of a microfluidic device before and after packing magnetic microparticles, respectively, according to an embodiment of the present invention;

FIG. 2 is a three-dimensional image of a structure illustrated in FIGS. 1A and 1B, according to an embodiment of the present invention;

FIG. 3 is a sectional view taken along a line III-III of FIG. 2, according to an embodiment of the present invention;

FIGS. 4 and, 5 are microphotographic images of magnetic microparticles used in performing an immunoassay using a microfluidic device according to an embodiment of the present invention;

FIG. 6 is a sequence of photographic images illustrating a procedure of packing magnetic microparticles using a microfluidic device in temporal order according to an embodiment of the present invention; and

FIG. 7 is a sequence of photographic images illustrating a procedure of performing an immunoassay using a microfluidic device in temporal order according to an embodiment of the present invention.

### DETAILED DESCRIPTION OF THE INVENTION

Hereinafter, the present invention will now be described in detail with reference to the accompanying drawings.

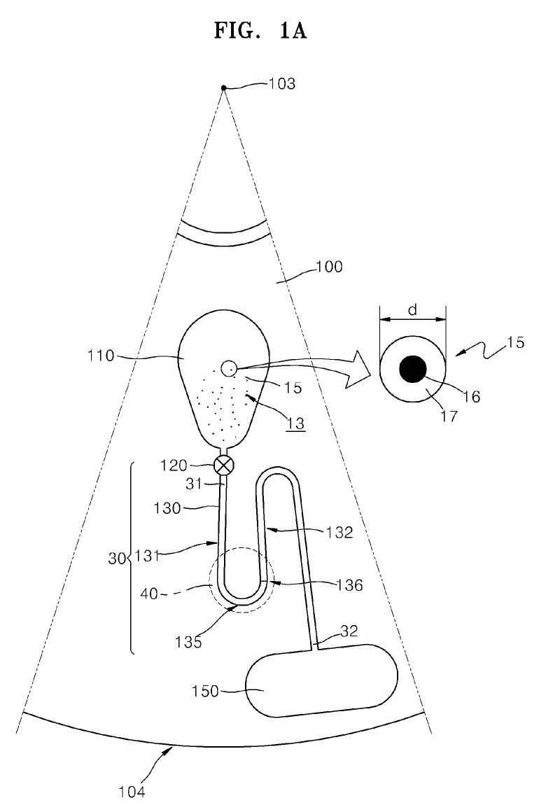

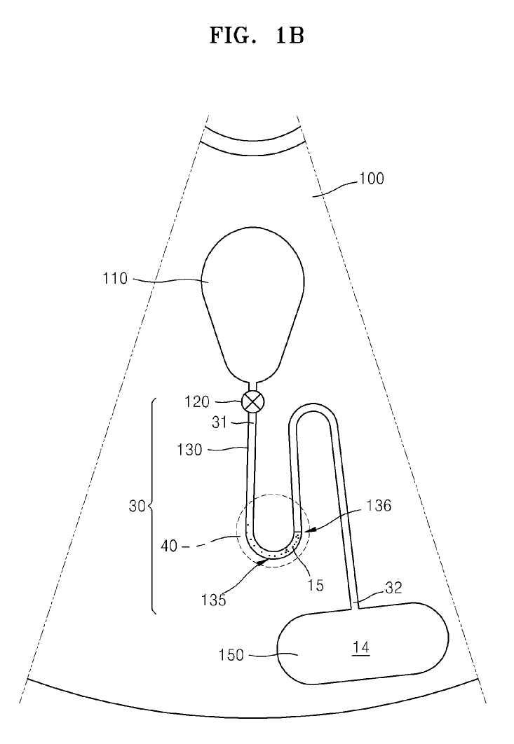

FIGS. 1A and 1B are plan views of a microfluidic device before and after packing magnetic microparticles, respectively, according to an exemplary embodiment of the present invention.

Referring to FIGS. 1A and 1B, the microfluidic device includes a rotating plate 100. The rotating plate 100 has a rotation center 103 and a periphery 104. A magnetic microparticle-packing unit 30 according to an embodiment of the present invention is disposed on the rotating plate 100. In one exemplary embodiment, the rotating plate 100 has an upper layer and a lower layer, and the upper and lower layers are joined together to define channels, chambers, flow passages, valves and others, which are described below. The structures may be formed in one of the upper and lower layers. The rotating plate 100 is an example of a rotary body that controllably rotates, but the present invention is not limited thereto.

The microfluidic device also includes a microfluidic channel 130 that includes an inlet 31 and an outlet 32 and provides a flow passage through which a fluid flows. In an aspect of the present invention, the fluid flows through the passage due to a pressure difference between the inlet 31 and the outlet 32. For this purpose, the distance from a rotation center 103 to the inlet 31 is smaller than that from the rotation center 103 to the outlet 32. When the rotating plate 100 rotates, a pressure difference between the inlet 31 and the outlet 32 is generated by centrifugal force. The inlet or inlet port 31 is in fluid communication with a sample chamber 110. The outlet or outlet port 32 is in fluid communication with a waste chamber 150.

The microfluidic channel 130 includes a curved portion or region 135, in which a fluid passage extends away from the rotation center 103 of the rotating plate 100 and then turns back toward the rotation center 103 of the rotating plate 100. Thus, the outer boundary of the curved portion 135 faces the periphery 104 of the rotating plate 100. The micro channel 130 may comprise a first flow channel 131 formed between the inlet 31 and the curved portion and a second flow channel 132 formed between the curved portion and the outlet 32. The microfluidic channel 130 may further include a bottleneck or contraction portion 136 where the flow passage is constricted in its width or diameter. The bottleneck portion 136 is situated proximate to the curved portion 135, usually behind the curved portion 135 in the direction of the flow of a fluid in the channel. That is, the curved portion 135 is situated between the curved portion 135 and the second flow passage 132. The bottleneck portion 136 allows magnetic microparticles 15 to be contained in the curved portion 135 by slackening a flow of a sample 13 which is mixed with the magnetic microparticles 15.

A magnet 40 may be disposed at a position adjacent to the curved portion 135 such that the magnet 40 applies a magnetic force to hold the magnetic microparticles 15 in the curved portion 135. The magnet 40 may be disposed in front of the bottleneck portion 136.

The microfluidic device according to the current embodiment of the present invention includes a first chamber 110 (“sample chamber”) and a second chamber 150 (“waste chamber”) disposed on the rotating plate 100. The sample chamber 110 is disposed closer to the rotation center 103 of the rotating plate 100 than the waste chamber 150. The microfluidic channel 130 is disposed between the sample chamber 110 and the waste chamber 150 to connect the sample chamber 110 and the waste chamber 150. The microfluidic channel is in fluid communication with the sample chamber 110 at one end and the waste chamber 150 at the other end. The curved portion 135 is disposed such that an outer boundary of the curved portion 135 faces the periphery of the rotating plate 100. The curved portion 135 is disposed in the microfluidic channel 130 between the inlet 31 and the outlet 32. Therefore, the microfluid channel 130 may include a first flow passage 131, which is formed between the inlet 31 and the curved portion 135, and a second flow passage 132, which is formed between the curved portion 135 and the outlet 32. The bottleneck portion 136, where the internal dimension of the flow passage is reduced, may be disposed in an end of the curved portion 135, i.e., between the curved portion 135 and the second flow passage 132.

The packing unit of the present invention may include a magnet to provide magnetic force which holds microparticles 15 in the curved portion 135. The magnet 40 may be disposed at any location on or under the rotating plate or in the rotating plate as long as it effectively holds microparticles 15 in the curved portion 135. For example, the magnet 40 may be disposed on the bottom surface or the upper surface of the rotating plate 100 at a location where the curved portion 135 is situated, or both on and under the curved portion 135. When the magnet 40 is disposed on the bottom surface of the rotating plate 100 and the upper layer of the rotating plate 100 is clear, it is easy to observe a surface of the magnetic microparticles 15 packed in the microfluidic channel 130.

The magnet 40 may be a permanent magnet or an electromagnet. A permanent magnet may be used as it has a high magnetic flux density and a small size, thus having excellent portability. Meanwhile, the magnet 40 may be ring-shaped. That is, a single belt of the magnet 40 (not shown) is situated on the upper or bottom surface of the rotating plate 100, along a trace of a rotation of the curved portion 135.

A valve 120 may be disposed between the inlet 31 of the microfluidic channel 130 and the sample chamber 110 to control a flow of fluid. The valve 120 may be, for example, a capillary valve or a hydrophobic valve that is open and closed by controlling the rotation of the rotating plate 100.

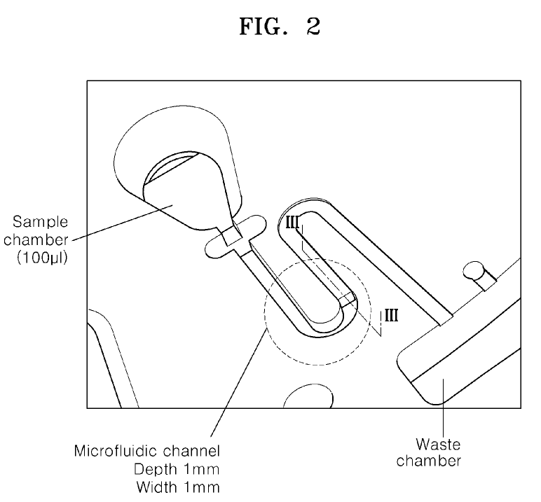

An operation of the microfluidic device illustrated in FIGS. 1 and 2 according to an embodiment of the present invention will now be described. The sample 13 and the magnetic microparticles 15 are introduced into the sample chamber 110. The sample 13 and the magnetic microparticles 15 may be mixed together, prior to the introduction into the sample chamber 110. Alternatively, they are brought to be in contact with each other in the sample chamber 110. When the valve 120 is open and the rotating plate 100 rotates, the pressure increases around the inlet 31 of the microfluidic channel 130 in which fluid is collected by a centrifugal force and the pressure decreases around the outlet 32 of the microfluidic channel 130 through which fluid flows out by a centrifugal force, which allows the sample 13 to flow in the microfluidic channel 130.

When the sample 13 containing the magnetic microparticles 15 reaches the curved portion 135, the speed of the magnetic microparticles 15 is reduced at the bottleneck portion 136 and the magnetic microparticles 15 may be held in the curved portion 135 by the magnetic force of the magnet 40 (refer to FIG. 1B). Also, as the fluid moves against centrifugal force toward the outlet 32, once it passes through the curved portion 135, the magnetic microparticles 15, which have generally a higher density than the fluid sample 13, are affected by a centrifugal force to a greater degree than the sample 13, and thus hardly move back towards the rotation center of the rotating plate 100. Also, the bottleneck portion 136 serves as a barrier that slows down the movement of magnetic microparticles. Thus, the magnetic microparticles 15 are separated from the sample 13 and contained in the curved portion 135, and a remaining waste 14 is discharged to the waste chamber 150 through the outlet channel 132.

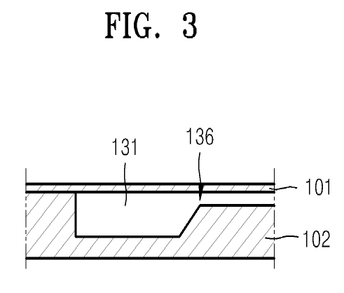

FIG. 2 is a three-dimensional image of the microfluidic illustrated in FIGS. 1A and 1B, according to an embodiment of the present invention. Intaglio structures composing the sample chamber 110, the microfluidic channel 130, and the waste chamber 150 are formed in a lower layer of the rotating plate 100. A transparent upper layer (not shown) may be bonded to the lower layer by an adhesive, heat or other means, which are known in the art. FIG. 3 is a sectional view taken along a line III-III of FIG. 2, according to an embodiment of the present invention. A lower layer 102 of the rotating plate, a upper layer 101, and the inlet channel 131 and the bottleneck portion 136 formed in the lower layer 102 are illustrated in FIG. 3. However, the present invention may be embodied in various shapes, for example, the microfluidic device as above may be disposed in the upper layer 101. Also, it is possible to fabricate the upper layer 101 to define some of these structures and the lower layer 102 to define the remaining structures of the microfluidic device. The methods of forming the chambers, channels, inlet/outlet ports and bottleneck portions are known in the art. For example, lithographic patterning and etching, or stamping may be used to fabricate the device having the structures.

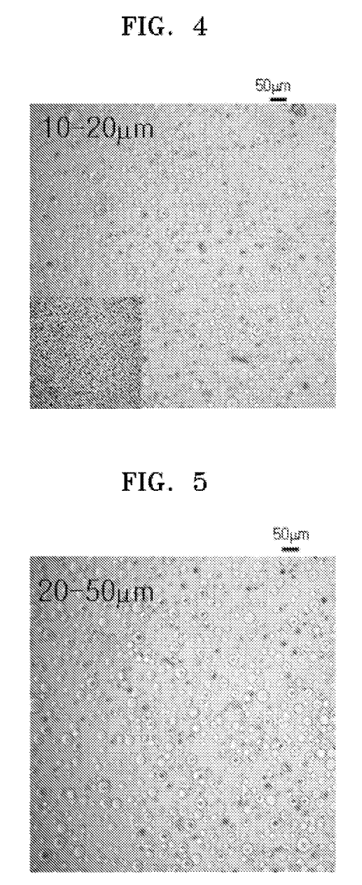

FIGS. 4 and 5 are microphotographic images of magnetic microparticles used in performing an immunoassay using a microfluidic device according to an embodiment of the present invention. FIG. 4 illustrates magnetic microparticles having a diameter of approximately 10 to 20 μm and FIG. 5 illustrates magnetic microparticles having a diameter of approximately 20 to 50 μm. The magnetic microparticles 15 (refer to an enlarged view illustrated in FIG. 1A) include a core 16 that is a magnetic particle and a shell 17 that is a coating layer surrounding the core 16. A diameter d of the magnetic microparticles 15 may range from 10 to 50 μm. While the figures show the bead shape of the microparticles, the “microparticle” includes a particle having a micro or nano meter size in its average diameter. The magnetic microparticles may have different shapes including, but not limited to, beads, tubes, or plates. In an exemplary embodiment, they are beads or 2-dimensional strips.

The magnetic microparticles 15 are relatively large-sized, compared to the microparticles having a diameter less than 10 μm, which are commonly used in microfluidic devices. If the diameter of the magnetic microparticles 15 is too small, the sample 13 flowing through the packed magnetic microparticles 15 experiences a significant pressure drop. Also, the operation efficiency of a surface treatment of the magnetic microparticles 15, for example, washing, attachment of biochemical substrates, or the like, may decrease as the diameter of the microparticles reduces. On the other hand, if the diameter of the magnetic microparticles 15 is too large, the porosity of the packed magnetic microparticles 15 increases, which causes a poor contact of biomolecules contained in the sample 13 with the magnetic microparticles 15, and thus are not suitable for performing a desired assay.

The core 16 of the magnetic microparticles 15 may be formed of any magnetic particle and have a diameter of several tens of nm to several μm. In particular, the core 16 may include at least one material selected from the group of ferromagnetic metals consisting of Fe, Ni, Cr and oxides thereof.

The shell or coating 17 of the magnetic microparticles 15 may be formed of a nonmagnetic material that does not shield a magnetic field. Also, since the shell 17 has many chances to contact a biological sample, the shell 17 may be biologically inert to prevent an unnecessary biochemical reaction. The shell 17 may be formed of biologically inert polymers which include, but are not limited to, styrene, agarose, dextran, polyethylene glycol (PEG), etc. as a material that satisfies such a condition.

The surface of the shell 17 may be biologically activated for nonspecific or specific binding with target biomolecules contained in samples. The biological activation includes a chemical surface modification. For example, the surface of the shell 17 may be modified with silica such that a target nucleic acid is nonspecifically bound to the surface of the shell 17 of the magnetic microparticles 15. In this case, the target nucleic acid may be extracted or purified by contacting a sample (e.g., cell lysis solution) which contains the target nucleic acid with the magnetic microparticles 15, allowing the target nucleic acid to be coupled to the surface of the magnetic microparticles 15. The cell lysis solution may be mixed with a chaotropic salt, NaOH solution, etc.

The surface of the shell 17 may be modified such that a target molecule can be specifically attached thereto. The shell 17 may include at least one probe selected from the group consisting of an antibody, antigen, nucleic acid molecule, biotin, protein, amino group (NH₂—), and carboxyl group (COOH—) that can be specifically coupled with a target molecule to be detected in the surface of the shell 17. Since the shell 17 is formed of a biologically inert material, the surface of the shell 17 may be previously coated with a material such as an epoxy, streptavidine, etc. to form the probe. When the magnetic microparticles 15 are surface-modified with an antibody (that is, microparticles 15 have an antibody probe), the magnetic microparticles 15 are useful for detecting a target antigen of low concentration from a small amount of a sample, because the antibody can selectively capture the target antigen.

Hereinafter, an exemplary method of fabricating magnetic microparticles to be used for a microfluidic device for an immunoassay according to an embodiment of the present invention will be briefly described.

Fabrication of Magnetic Nanoparticles

A 20 ml solution of 0.4 M FeCl₂ and 0.25 M FeCl₃ was prepared and stirred for 30 minutes at a room temperature. 20 ml of 5 M NaOH solution was slowly added to the aforementioned solution and the resulting mixture was stirred for 10 minutes to form magnetic particles. The magnetic particles were washed by distilled water until they were neutralized. The magnetic particles were thoroughly dried at 80° C. and then a dry weight of the magnetic particles was determined.

Fabrication of Magnetic Agarose Beads

4 g of Agarose was added to 100 ml of distilled water and then melted using a microwave oven. 4 g of magnetic particles were added to the distilled water and then stirred well. 5 g of Ethyl cellulose was melted in 100 ml of toluene and then stirred and thoroughly melted for 2 hours or more at 60° C. The aforementioned agarose solution was slowly added to the ethyl cellulose solution and then stirred for 30 minutes. The resulting solution was cooled down to a temperature of 25° C., the resulting solution was washed with 100% ethyl alcohol twice, 70% ethyl alcohol twice, and with distilled water several times.

The generated beads were observed using a microscope. The agarose beads fabricated using the aforementioned method had a diameter of approximately 10 to 50 μm.

Cross-Linking and Activation of Magnetic Agarose Beads

100 ml of Agarose beads were added to 100 ml of 0.5 N sodium hydroxide, and 10 ml of 1,4-butanediol diglycidyl ether or epichlorohydrin were added thereto. The resulting mixture was stirred and reacted for 8 hours at 25° C. The resulting solution was washed with 5 L of distilled water. The number of epoxy group molecules introduced in the beads was measured using HCI titration of epoxy molecules dissociated and generated in 3M Na₂S₂O₃ solution, and the beads were kept at 4° C.

Conjugation of an Antibody

A method of conjugating an antibody to the epoxy-activated magnetic agarose beads will be described. An antibody diluted at a concentration of 0.1 to 1 mg/ml (50 mM sodium carbonate buffer) was mixed with beads, washed by 50 mM sodium carbonate buffer (pH 9.0) several times and reacted for 16 hours or more at 4° C. The amount of the antibody remaining in the reaction solution was measured by determining the amount of a protein using a Bradford method and converted it to measure an amount of the antibody conjugated to the beads. The reacted beads were washed with 50 mM sodium carbonate buffer several times and then processed with 1 M glycine solution for 12 hours to remove the active group without the antibody attached. The beads were neutralized by washing by PBS several times and kept at 4° C. The activation of the antibody conjugated to the beads was observed by an ELISA method using a plate coated with an antigen.

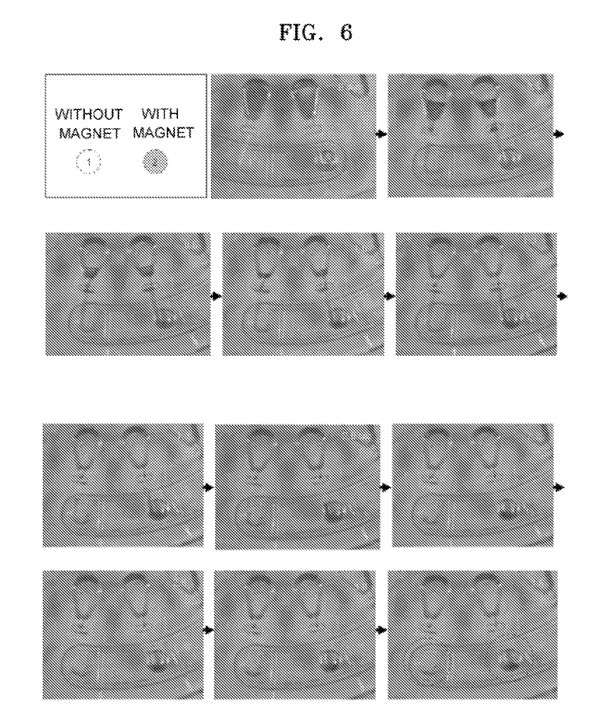

FIG. 6 is a sequence of photographic images illustrating a procedure of packing magnetic beads using a microfluidic device in temporal order according to an embodiment of the present invention. A microfluidic device having a magnetic bead-packing unit according to an embodiment of the present invention is illustrated on the right side of each of the photographic images and a microfluidic device without a magnet is illustrated on the left side to compare to the present invention. The numerical number illustrated in a right upper portion of each of the photographic images represents time elapsed in units of seconds.

The magnetic beads and sample are injected into the sample chamber and mixed. While the rotating plate of the CD-shaped microfluidic device rotates, the valve connected with the outlet of the sample chamber is open. When the sample mixed with the magnetic beads passes through the microfluidic channel and reaches the curved portion, the magnetic beads are packed in the curved portion due to magnetic force and centrifugal force, and the sample waste moves to the waste chamber. After about 3 seconds, it can be found that the magnetic beads (dark color) are packed in the curved portion adjacent to the magnet.

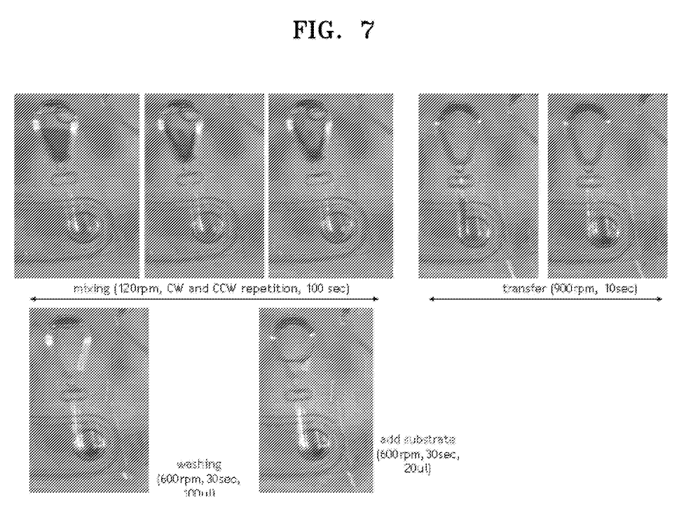

FIG. 7 is a sequence of photographic images illustrating a procedure of performing an immunoassay using a CD-shaped microfluidic device in temporal order according to an embodiment of the present invention. The sample including an antigen was injected into the sample chamber, and the prepared magnetic beads were mixed therein. The mixing operation can be performed by repeatedly rotating the rotating plate of the microfluidic device in both directions. In the present embodiment, the rotating plate was repeatedly rotated at 120 rpm for 100 seconds in clockwise and counterclockwise directions. The magnetic beads may be mixed with the sample prior to the introduction into the sample chamber.

Next, the sample mixed with the magnetic beads moved through the microfluidic channel due to centrifugal force. In the present embodiment, the rotating plate was rotated at 900 rpm for 10 seconds in any direction. As illustrated in FIG. 7, the sample mixed with the magnetic beads moved through the microfluidic channel and the magnetic beads are packed in the curved portion.

Next, the buffer solution was injected into the sample chamber and the packed magnetic beads were washed as the buffer solution moved along the channel due to centrifugal force. In the present embodiment, the rotating plate spun at 600 rpm, and the packed magnetic beads were washed using 100 μl of the buffer solution for 30 seconds. Next, 20 μl of biochemical substrate for detecting an immune reaction may be injected separately or simultaneously in the same way as the washing operation to attach the biochemical substrate to a surface of the packed magnetic beads. The biochemical substrate may specifically or non-specifically bind to the target substance which is coupled to the probe of the magnetic beads. The biochemical substrate may have a marker or label which allows detection of the immune reaction. The marker or label may be a radioisotope, fluorescent group or a color-generating group. The immune reaction may be detected using a method of measuring the fluorescence, color, or radioisotope depending on the kind of the label or marker of the biochemical substrate. The immune reaction may be quantitatively determined.

According to the present invention, magnetic microparticles can be effectively packed using a centrifugal force and a magnetic force in a microfluidic structure disposed in a rotary body. That is, a magnetic microparticle-packing unit and a microfluidic device including the same have simplified structures and thus the microfluidic device can be miniaturized, can reduce driving power, and can derive a high efficiency immune reaction.

Also, since the immunoassay method according to the present invention uses the microfluidic device and adequately-sized magnetic microparticles according to the present invention, the immunoassay method can be conveniently performed with low driving energy.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A magnetic microparticle-packing unit comprising:
a rotary body which controllably rotates and comprises a rotation center and a periphery;
a microfluidic channel disposed on the rotary body, said microfluidic channel comprising an inlet, an outlet, a curved portion, a first flow passage formed between the inlet and the curved portion, and a second flow passage formed between the curved portion and the outlet, wherein the inlet, the outlet, the curved portion, the first flow passage and the second flow passage each are in fluid communication with the other; wherein a distance from the rotation center of the rotary body to the inlet is smaller than that from the rotation center of the rotary body to the outlet, and a flow direction in the flow passage is from the inlet to the outlet; and wherein the curved portion is formed in such a way that the flow passage first extends away from the rotation center of the rotary body and then turns toward the rotation center of the rotary body; and
a magnet which is disposed so as to apply a magnetic force to the curved portion.

2. The magnetic microparticle-packing unit of claim 1, wherein the microfluidic channel further comprises a bottleneck portion in which the internal dimension of the flow passage is reduced.

3. The magnetic microparticle-packing unit of claim 2, wherein the bottleneck portion is disposed between the curved portion and the second flow passage.

4. The magnetic microparticle-packing unit of claim 1, wherein the magnet is affixed to the rotary body at a position adjacent to the curved portion.

5. The magnetic microparticle-packing unit of claim 1, wherein the magnet is in the shape of a ring which has a radius corresponding to the distance from the rotation center of the rotary body to the curved portion of the microfluidic channel, and is disposed outside the rotary body along the rotation trace of the curved portion.

6. The magnetic microparticle-packing unit of claim 1, wherein the curved portion is U-shaped or V-shaped such that an outer boundary of the curved portion faces the periphery of the rotating body.

7. The magnetic microparticle-packing unit of claim 1, wherein the rotary body is a compact disc-shaped rotating plate.

8. The magnetic microparticle-packing unit of claim 1, wherein the rotary body comprises a upper layer and a lower layer, which are joined together in such a way to define the microfluidic channel.

9. A microfluidic device comprising:
a rotary body which controllably rotates and comprises a rotation center and a periphery;
a microfluidic channel disposed on the rotary body, said microfluidic channel comprising an inlet, an outlet, a curved portion, a first flow passage formed between the inlet and the curved portion, and a second flow passage formed between the curved portion and the outlet, wherein the inlet, the outlet, the curved portion, the first flow passage and the second flow passage each are in fluid communication with the other; wherein a distance from the rotation center of the rotary body to the inlet is smaller than that from the rotation center of the rotary body to the outlet, and a flow direction in the flow passage is from the inlet to the outlet; and wherein the curved portion is formed in such a way that the flow passage first extends away from the rotation center of the rotary body and then turns toward the rotation center of the rotary body; and
a magnet which is disposed so as to apply a magnetic force to the curved portion;
a first chamber which is in fluid communication with the microfluidic channel through the inlet of the microfluidic channel; and
a second chamber which is in fluid communication with the microfluidic channel through the outlet of the microfluidic channel.

10. The microfluidic device of claim 9, further comprising a bottleneck portion which is disposed in the microfluidic channel, wherein the bottleneck portion has an internal dimension which is smaller than the other portions of the microfluidic channel.

11. The microfluidic device of claim 10, wherein the bottleneck portion is disposed between the curved portion and the second flow passage.

12. The microfluidic device of claim 9, wherein the magnet is affixed to the rotary body at a position adjacent to the curved portion.

13. The microfluidic device of claim 9, wherein the magnet is in the shape of a ring which has a radius of rotation corresponding to the distance from the rotation center of the rotary body to the curved portion of the microfluidic channel, and is disposed outside the rotary body along the rotation trace of the curved portion.

14. The microfluidic device of claim 9, wherein the curved portion is U-shaped or V-shaped such that an outer boundary of the curved portion faces the periphery of the rotating body.

15. The microfluidic device of claim 9, wherein the rotary body is a compact disc-shaped rotating plate.

16. The microfluidic device of claim 9, further comprising a valve to control a flow of fluid, said valve being disposed between the sample chamber and the inlet of the microfluidic channel.

17. The microfluidic device of claim 15, wherein the rotary body comprises a upper layer and a lower layer, which are joined together in such a way to define the microfluidic channel, the first chamber, and the second chamber.

18. An immunoassay method using a microfluidic device, comprising:
providing a fluid sample to be assayed for a target substance and magnetic microparticles which are capable of capturing the target substance; and
allowing the magnetic microparticles be in contact with the fluid sample in the microfluidic device,
wherein the microfluidic device comprises
a rotary body which controllably rotates and comprises a rotation center and a periphery;
a microfluidic channel disposed on the rotary body, said microfluidic channel comprising an inlet, an outlet, a curved portion, a first flow passage formed between the inlet and the curved portion, and a second flow passage formed between the curved portion and the outlet, wherein the inlet, the outlet, the curved portion, the first flow passage and the second flow passage are each in fluid communication with the other; wherein a distance from the rotation center of the rotary body to the inlet is smaller than that from the rotation center of the rotary body to the outlet, and the fluid sample and magnetic microparticles flow in a direction from the inlet to the outlet; and wherein the curved portion is formed in such a way that the flow passage first extends away from the rotation center of the rotary body and then turns toward the rotation center of the rotary body;
a magnet which is disposed so as to apply a magnetic force to the curved portion;
a first chamber which is in fluid communication with the microfluidic channel through the inlet of the microfluidic channel; and
a second chamber which is in fluid communication with the microfluidic channel through the outlet of the microfluidic channel; and
wherein the fluid sample and the magnetic microparticles are contacted to each other in the first chamber, the magnetic microparticles are retained in the curved portion, and the fluid sample is discharged through the outlet.

19. The method of claim 18, wherein the diameter of the magnetic microparticle ranges from 10 to 50 μm.

20. The method of claim 19, wherein the magnetic microparticles comprise a core formed of a magnetic material and a shell formed of a nonmagnetic material on the surface of the core.

21. The method of claim 20, wherein the shell is formed of a biologically inert polymer material and a surface of the shell is modified to be biologically active.

22. The method of claim 21, wherein the shell is formed of a material selected from the group consisting of styrene, agarose, dextran, and polyethylene glycol (PEG).

23. The method of claim 21, wherein the surface of the shell is modified to have biomolecules which are bound thereto.

24. The method of claim 23, wherein the surface of the shell has at least one probe selected from the group consisting of an antibody, antigen, nucleic acid molecule, biotin, protein, amino group (NH₂—), and carboxyl group (COOH—).

25. The method of claim 20, wherein the surface of the shell is modified with silica.
